(19) United States
(12) Patent Application Publication
(10) Pub. No.: US 2014/0277124 A1
(43) Pub. Date: Sep. 18, 2014

(54) TISSUE ANCHORING AND DEPLOYMENT SYSTEMS

(71) Applicant: MimOSA Medical, Inc., Menlo Park, CA (US)

(72) Inventors: Michael H. ROSENTHAL, Menlo Park, CA (US); Donald A. GONZALES, San Antonio, CA (US); Sergio SALINAS, Redwood City, CA (US); Jose L. GARCIA, Fremont, CA (US)

(73) Assignee: MimOSA Medical, Inc., Menlo Park, CA (US)

(21) Appl. No.: 14/273,818

(22) Filed: May 9, 2014

Related U.S. Application Data
(63) Continuation of application No. 13/839,401, filed on Mar. 15, 2013.

(51) Int. Cl. A61B 17/04

(52) U.S. Cl. CPC A61B 17/0401; A61B 2017/0414
USPC 606/228; 606/232

(57) ABSTRACT

Tissue anchoring and deployment systems are described herein. Generally, an anchor housing may define a receiving channel within or along its periphery. A securement member which is adjustably slidable relative to the receiving channel and further defining a suture receiving channel along a portion of the member may also be used such that the suture receiving channel is aligned with an opening defined along a first surface. The securement member and a compression surface along the receiving channel are spaced apart from one another and form a suture compression interface. Additionally, a length of suture may also be used where the suture has a first portion positioned along the suture compression interface and a second portion passed through the suture receiving channel and opening along the first surface. The compression interface has a height which is sized to be relatively smaller than a diameter of the first portion of suture.

### CROSS-REFERENCE TO RELATED APPLICATIONS

This application is a continuation of U.S. patent application Ser. No. 13/839,401 filed Mar. 15, 2013, which is incorporated herein by reference in its entirety.

### FIELD OF THE INVENTION

The present invention relates generally to medical devices used for securing and approximating wounds and tissue regions. More particularly, the present invention relates to apparatus and methods for anchoring and securing tissue regions towards one another for wound repair and/or securing portions of tissue for various treatments.

### BACKGROUND OF THE INVENTION

Suturing tissues and performing suturing techniques in confined spaces remains a difficult challenge. The ability to tie knots requires a two-handed technique and is fraught with challenges especially when using endoscopic and/or laparoscopic instruments for treating, e.g., oropharyngeal tissues, or regions of the body where space constraints or exposed knots are an issue.

In addition to the difficulty in performing such techniques, the treatment outcomes are often less than ideal. For instance, when closing the oropharyngeal tissue with suture such as for an uvulopalatopharyngoplasty (UPPP) procedure, the suture typically tends to pull through the friable mucosa and may result in pain inflicted upon the patient due to the exposed or extruded suture knots.

Additionally, the time required can often exceed 20 minutes for the closure. The same problems are often seen in other procedures performed upon mucosal tissues such as for bowel anastomosis, closure of biopsy wounds, plications of the stomach, etc. This phenomenon is not only limited to mucosal tissue but also other areas where soft tissues are approximated or secured, such as for shoulder plications, etc.

Thus, there is a need for instruments and procedures which allow for the deployment of tissue securement devices which enable the securement of soft tissue regions, particularly in areas of the body where space is limited.

### BRIEF SUMMARY OF THE INVENTION

Tissue anchors which are optimally sized for tolerability within a patient's mouth and which are also configured to lock against suture without the need for tying knots or exposing terminal suture lengths. Moreover, such tissue anchors having a low-profile may also reduce any potential irritation associated with knots or suture tails interacting with surrounding tissues. Such tissue anchors may generally comprise an anchor housing which defines a first surface for presentation against a tissue surface, a second surface opposite to the first surface, and a periphery between the first and second surfaces, the anchor housing defining a receiving channel within or along the periphery. A securement member which is adjustably slidable relative to the receiving channel and further defining a suture receiving channel along a portion of the member may also be used such that the suture receiving channel is aligned with an opening defined along the first surface, wherein the securement member and a compression surface along the receiving channel are spaced apart from one another and form a suture compression interface. Additionally, a length of suture may also be used where the suture has a first portion positioned along the suture compression interface and a second portion passed through the suture receiving channel and opening along the first surface, wherein the suture compression interface has a height which is sized to be relatively smaller than a diameter of the first portion of suture.

In an example of using such tissue anchors, one method of loading a length of suture into an anchor may generally comprise providing the anchor housing which defines a first surface for presentation against a tissue surface, a second surface opposite to the first surface, and a periphery between the first and second surfaces, the anchor housing defining a receiving channel within or along the periphery. A length of suture having a first portion and a second portion may be placed within or along a suture receiving channel defined along a portion of a securement member such that the length of suture is partially engaged by the securement member to facilitate handling of the length of suture. The securement member may be adjustably slidable into the receiving channel such that the suture receiving channel is aligned with an opening defined along the first surface. The first portion of suture may be compressed within or along a suture compression interface formed between the securement member and a compression surface along the receiving channel spaced apart from one another, wherein the suture compression interface has a height which is sized to be relatively smaller than a diameter of the first portion of suture, and wherein the first portion of suture passes through the periphery between the first and second surfaces and the second portion of suture passes through the opening along the first surface.

In delivering and deploying the tissue anchors, an anchor delivery assembly may be used which may generally comprise an elongate member having a distal end with a cartridge receiving assembly attached thereto, a cartridge housing detachably insertable into the cartridge receiving assembly, the cartridge housing having at least one translatable member defining a tapered surface at a first end, the cartridge housing further defining a cartridge opening into which one or more tissue anchors are positionable, and a plunger translatable relative to the cartridge housing, wherein actuation of the plunger contacts the tapered surface of the translatable member and urges the translatable member into contact against the one or more tissue anchors, wherein the one or more tissue anchors each comprise an anchor housing which defines a first surface for presentation against a tissue surface, a second surface opposite to the first surface, and a periphery between the first and second surfaces, the anchor housing defining a receiving channel within or along the periphery.

Additionally, the assembly may also include a securement member which is adjustably slidable relative to the receiving channel and further defining a suture receiving channel along a portion of the member such that the suture receiving channel is aligned with an opening defined along the first surface, wherein the securement member and a compression surface along the receiving channel are spaced apart from one another and form a suture compression interface, and a length of suture having a first portion positioned along the suture compression interface and a second portion passed through the suture receiving channel and opening along the first surface, wherein the suture compression interface has a height which is sized to be relatively smaller than a diameter of the first portion of suture.

A length of suture may be configured within the tissue anchor to enter in an axial direction relative to the anchor housing while exiting the anchor housing in a peripheral or lateral direction. As the suture length is passed through the anchor housing, a portion of the suture may be compressed along the lateral direction. Additionally, the anchor may have a male component which may lock into a female receiving channel such that interaction between the male component and female component provides for multiple locations of securement for the suture by the tissue anchor. For instance, the interaction between the male and female components may provide suture securement by providing for a compression length of a portion of the suture, a tortuous passageway through the anchor housing, and a wedged or compressed entry point forcing the suture between the male and female components.

Moreover, the tissue anchors are further designed to provide for increased strength for ensuring suture securement without the anchor housing failing. The ability of the anchor housing to compress the suture within the anchor body may be due in part to the preservation of a uniform structure of the anchor housing. The anchor housing into which the male component is inserted may be designed to preserve a uniform structure along the different planes of the anchor body. That is, no part of the anchor housing may be formed with an opening profile that extends through the entire structure of the anchor housing thus providing for increased strength of the anchor when secured to the suture.

Furthermore, the tissue anchor may be formed to be biodegradable or bioabsorbable such that the tissue anchor may dissolve or become absorbed by the patient's body over a predetermined period of time. The ability of the anchors to be biodegraded or bioabsorbed may provide particular challenges because of the need to hold or secure approximated tissue portions for extended periods of time. Typically, biodegradable or bioabsorbable materials may not provide the sufficient material properties for securing a suture length. However, because of the configuration of the male and female components and the relative interaction between the two, various biodegradable materials may be used to fabricate the tissue anchor, e.g., polylactic acid (PLA), poly-DL-lactide (PLLA), polyglycolic acid (PGA), Poly (Î-caprolactone) (PCL), polyether sulfone (PES), polydioxanone (PDS), co-polymers of the materials, etc.

Additional variations of tissue anchors and deployment methods which may be used with the instruments and methods described herein may be found in further detail in U.S. Prov. 61/710,516 filed Oct. 5, 2012 and 61/698,279 filed Sep. 7, 2012, each of which is incorporated herein by reference in its entirety for any purpose.

One variation of an anchor assembly may have the anchor housing formed as a housing which defines an opening along its periphery which opens into a receiving channel extending from the opening and at least partially into or through the anchor housing. The anchor housing may also define a notch or shoulder extending at least partially along the periphery over the opening. The securement member may be configured as a planar member having a width sized to correspond to the receiving channel and having a height which is slightly less than a height of the channel to accommodate a length of suture. The securement member may be slidingly introduced through the opening and into the receiving channel such that a notch at a distal end of the securement member interfits with a member within the housing in a corresponding manner to provide for added stability between the two structures.

The securement member may further define a suture receiving notch extending towards a central portion of the member and having an optional retaining arm extending over the notch to facilitate side-loading insertion of a suture length into the notch prior to locking insertion of the member into the receiving channel.

The anchor housing may be sized into various dimensions so long as the anchor assembly is suitably tolerable for placement within the patient's body, such as within the patient's mouth where the patient is continually exposed to interaction with the anchor housing. Moreover, the anchor housing may be configured into various shapes which may present an atraumatic surface to the surrounding tissue of the patient. In one configuration, the anchor housing may be configured into a circular shape having, e.g., a diameter of 3.5 mm or up to 5.0 mm and a height of 1.5 mm or up to 3.0 mm, although other suitable sizes may be used so long as the anchor housing is able to provide the securement force for the suture.

A first surface may be defined along a portion of the anchor housing which may contact against the tissue surface. The first suture may define a suture locking channel which may extend from the opening along the periphery of the anchor housing and extend along the receiving channel towards a central portion of the anchor housing. A second surface opposite to the first surface may present a planarly uniform and uninterrupted surface for providing additional strength to the anchor housing.

To lock or secure a length of suture to the anchor assembly, a length of suture may be initially inserted into the suture receiving notch defined along the securement member. The securement member may then be inserted into the receiving channel of the anchor housing. Optionally, the securement member may be inserted into the anchor housing through various interfacing mechanisms. For instance, the securement member may comprise a separate component which may be longitudinally inserted into the anchor housing while in other variations, the securement member may be pivotably attached to the anchor housing such that engagement between the two members may be accomplished by rotating the securement member into locking contact within the anchor housing.

As the suture is urged into the anchor housing, a first portion of the suture may be squeezed between the apposed surfaces of the securement member and the receiving channel forming a suture compression interface such that the first portion of suture is compressed along a plane, e.g., parallel to a plane defined by the anchor housing. With the first portion of suture compressed within the suture compression interface of the enclosed anchor assembly, an interference fit may be created between the first portion and the anchor housing.

Moreover, the height of the suture compression interface between the securement member and the receiving channel may be varied by altering the respective dimensions depending upon the desired degree of compression along the suture and the diameter of the suture used. With the variability, the height may be dimensioned to be no greater than 75% of the uncompressed diameter of the first portion of suture in one variation or no greater than 90% of the uncompressed diameter of the first portion in another variation. Additionally, the length of the suture compression interface may also be varied through the anchor housing to range from, e.g., 0.5 mm to 1.75 mm.

With the first portion extending from the opening about the periphery of the anchor housing and along the securement member, the first portion of suture may then curve or bend through the suture receiving notch within the anchor housing such that a second portion of suture passes through the first surface of the anchor housing. The first portion of suture may accordingly be orthogonal to or form an angle with the second portion of suture, e.g., 60 degrees and up to 135 degrees. The suture passes through the first surface along a longitudinal axis of the anchor housing, forms a tortuous passageway around notch, is compressed along the suture compression interface, and then exits along the periphery of the anchor assembly.

To accommodate the second portion of suture passing through the first surface, the anchor housing may define a slot along the first surface which extends from the opening towards a center portion of the anchor housing. The slot may have a width ranging from, e.g., 40% to 50%, of a width or diameter of the anchor housing. When the securement member is urged entirely into the receiving channel, the slot may form an additional compression point with the notch of the securement member against the suture. The resulting compression and pinching of the suture through the anchor housing may result in a resistive pull force upon the suture of 0.125 lb and up to 3.5 lb. Moreover, even with the use of the biodegradable or bioabsorbable materials used in fabricating the anchor assembly, having the second surface present a uniform and continuous surface helps to prevent or inhibit deformation of the anchor housing upon application of the load to the suture which also may help to enable a significant compression locking force at the interface. Additionally, the distal portion of the receiving channel is also configured to limit expansion of the anchor housing upon application of the load upon the suture.

The length of the suture may be initially inserted into notch and then the securement member may be advanced distally into the receiving channel once the suture is ready to be secured. The first portion of suture may become compressed along the suture compression interface formed between the upper surface of the securement member and the apposed inner surface of the receiving channel such that the first portion of suture is compressed to a reduced thickness, e.g., up to 75%, which is less than the uncompressed suture diameter. The first portion of suture may then pass through the notch of securement member such that the second portion of suture passes through the first surface of the anchor housing. The suture may thus be compressed along the interface, secured via the tortuous passageway around notch, and also pinched between the notch and slot defined along the first surface.

In deploying one or more of the tissue anchors described herein, a deployment instrument may be used. The deployment instrument may generally comprise a handle having an actuation plunger attached. An elongate shaft may extend from the handle with a cartridge engagement housing positioned at its distal end. An anchor deployment cartridge may be removably attached to the engagement housing for aligning the tissue anchors to be deployed. Each of the deployment cartridges may contain a single tissue anchor or two or more tissue anchors aligned and engaged for deployment into the tissue region. Once the tissue anchors have been deployed or spent from the deployment cartridge, the cartridge may be ejected from the engagement housing and a new cartridge having additional tissue anchors may be engaged to the housing.

As the cartridge may generally hold one or two tissue anchors, multiple cartridges may be utilized with the deployment instrument, e.g., when suturing a portion of tissue with multiple interrupted sutures. Each individual cartridge may be positioned within a cartridge slot adjacent to one another in a circular manner. The cartridge assembly may be rotated during use to facilitate the insertion of a cartridge into the engagement housing of the deployment instrument.

The individual cartridges positioned upon the assembly may be specially marked or have some visual indicator to optionally indicate the first cartridge to be used and/or a terminal cartridge to be used, if so desired, where the initial cartridge or terminal cartridge may contain a single tissue anchor or specialized tissue anchor. The individual tissue anchors within the initial and/or terminal cartridge may also optionally incorporate some visual indicator, such as coloring, to indicate the initial tissue anchor (e.g., colored green) used in a procedure and/or a terminal tissue anchor (e.g., colored red) used in the procedure.

### BRIEF DESCRIPTION OF THE DRAWINGS

FIGS. 1A and 1B show one variation of an anchor assembly with an anchor housing and securement member adjustably separated from one another.

FIG. 2 shows a top view of the securement member partially advanced into the anchor housing.

FIG. 3 shows a perspective view of a suture locked within a tissue anchor such that an interference fit is created between the suture and the anchor housing.

FIG. 4A shows a cross-sectional side view of a securement member prior to being inserted into the anchor housing.

FIG. 4B shows a cross-sectional side view of a suture positioned within or along a suture compression interface between the securement member and the anchor housing.

FIG. 4C shows a cross-sectional side view of another variation where the suture may be alternatively formed into a tortuous passageway between the distal end of securement member and the end of the receiving channel.

FIG. 5 shows a perspective view of one variation of a deployment instrument.

FIG. 6 shows a perspective view of one variation of a cartridge which may house the one or more tissue anchors.

FIG. 7 shows a perspective view of a cartridge assembly configured as a carousel.

FIGS. 8A to 8D illustrate an example of how the deployment instrument may be used with the cartridge assembly.

FIGS. 9A to 9F illustrate perspective views showing one example of how the cartridge may be used with the deployment instrument to suture a region of tissue.

FIGS. 10A to 10F illustrate perspective views of yet another variation of the tissue anchor which may have a rotatable locking feature.

FIGS. 11A to 11D illustrate perspective and views of the rotatably locking tissue anchor in its locked configuration.

FIGS. 12A to 12C illustrate side and cross-sectional side views of the rotatably locking tissue anchor.

FIG. 13 shows a perspective view of one example of multiple tissue anchors deployed in a tissue region such as the oral cavity of a patient.

### DETAILED DESCRIPTION OF THE INVENTION

In suturing and plicating soft tissues, particularly along tissue regions located in areas of the body where space is limited (e.g., gastrointestinal tissues, oropharyngeal, laryngeal, vascular tissues, etc.), tissue anchors which are optimally sized for tolerability within a patient's mouth and which are also configured to lock against suture without the need for tying knots or exposing terminal suture lengths may be used to form a tortuous passageway through the anchor. The instruments and tissue anchors may also produce tissue closures with reduced procedure times, create more durable closures, and create improved patient outcomes with reduced pain and improved healing.

A length of suture may be configured within the tissue anchor to enter in an axial direction relative to the anchor housing while exiting the anchor housing in a peripheral or lateral direction. As the suture length is passed through the anchor housing, a portion of the suture may be compressed along the lateral direction. Additionally, the anchor may have a male component which may lock into a female receiving channel such that interaction between the male component and female component provides for multiple locations of securement for the suture by the tissue anchor. For instance, the interaction between the male and female components may provide suture securement by providing for a compression length of a portion of the suture, a tortuous passageway through the anchor housing, and a wedged or compressed entry point forcing the suture between the male and female components.

Moreover, the tissue anchors are further designed to provide for increased strength for ensuring suture securement without the anchor housing failing. The ability of the anchor housing to compress the suture within the anchor body may be due in part to the preservation of a uniform structure of the anchor housing. The anchor housing into which the male component is inserted may be designed to preserve a uniform structure along the different planes of the anchor body. That is, no part of the anchor housing may be formed with an opening which extends through the entire structure of the anchor housing thus providing for increased strength of the anchor when secured to the suture.

Furthermore, the tissue anchor may be formed to be biodegradable or bioabsorbable such that the tissue anchor may dissolve or become absorbed by the patient's body over a predetermined period of time. The ability of the anchors to be biodegraded or bioabsorbed may provide particular challenges because of the need to hold or secure approximated tissue portions for extended periods of time. Typically, biodegradable or bioabsorbable materials may not provide the sufficient material properties for securing a suture length. However, because of the configuration of the male and female components and the relative interaction between the two, various biodegradable materials may be used to fabricate the tissue anchor, e.g., polylactic acid (PLA), poly-DL-lactide (PLLA), polyglycolic acid (PGA), Poly (Î-caprolactone) (PCL), poly-ether sulfone (PES), polydioxanone (PDS), co-polymers of the materials, etc.

As shown in the top views of FIGS. 1A and 1B, one variation of an anchor assembly 10 is shown with anchor housing 12 and securement member 14 separated from one another. Anchor housing 12 and securement member 14 may be fabricated from the same or similar materials such that one or both components are biodegradable or bioabsorbable, as mentioned above. The anchor housing 12 may be formed as a housing which defines an opening 16 along its periphery which opens into a receiving channel 26 extending from the opening 16 and at least partially into or through the anchor housing 12.

The anchor housing 12 may also define a notch or shoulder 18 extending at least partially along the periphery over the opening 16. The securement member 14 may be configured as a planar member having a width sized to correspond to the receiving channel 26 and having a height which is slightly less than a height of the channel 26 to accommodate a length of suture, as described in further detail herein. The securement member 14 may be slidingly introduced through the opening 16 and into the receiving channel 26 such that a notch 20 at a distal end of the securement member 14 interfits with a member 20′ within the housing 12 in a corresponding manner to provide for added stability between the two structures.

The securement member 14 may further define a suture receiving notch 22 extending towards a central portion of the member 14 and having an optional retaining arm 24 extending over the notch 22 to facilitate side-loading insertion of a suture length into the notch 22 prior to locking insertion of the member 14 into the receiving channel 26.

The anchor housing 12 may be sized into various dimensions so long as the anchor assembly 10 is suitably tolerable for placement within the patient's body, such as within the patient's mouth where the patient is continually exposed to interaction with the anchor housing 12. Moreover, the anchor housing 12 may be configured into various shapes which may present an atraumatic surface to the surrounding tissue of the patient. In one configuration, the anchor housing may be configured into a circular shape having, e.g., a diameter of 3.5 mm or up to 5.0 mm and a height of 1.5 mm or up to 3.0 mm, although other suitable sizes may be used so long as the anchor housing 12 is able to provide the securement force for the suture.

As further shown in the top view of FIG. 2, the securement member 14 is shown partially advanced into the anchor housing 12. In this variation, the suture receiving notch 22 may omit a retaining arm. A first surface 36 may be defined along a portion of the anchor housing 12 which may contact against the tissue surface. The first suture 36 may define a suture locking channel 40 which may extend from the opening 16 along the periphery of the anchor housing 12 and extend along the receiving channel 26 towards a central portion of the anchor housing 12, as indicated. A second surface 38 opposite to the first surface 36 may present a planarly uniform and uninterrupted surface for providing additional strength to the anchor housing 12.

To lock or secure a length of suture to the anchor assembly 10, a length of suture 30 may be initially inserted into the suture receiving notch 22 defined along the securement member 14. Securement member 14 may then be inserted into the receiving channel 16 of anchor housing 12. As the suture 30 is urged into the anchor housing 12, a first portion 32 of the suture may be squeezed between the apposed surfaces of the securement member 14 and the receiving channel 16 forming a suture compression interface 42 such that the first portion 32 of suture is compressed along a plane, e.g., parallel to a plane defined by the anchor housing 12, as shown in the perspective view of FIG. 3. With the first portion 32 of suture compressed within the suture compression interface 42 of the enclosed anchor assembly 10, an interference fit may be created between the first portion 32 and the anchor housing 12.

Moreover, the height of the suture compression interface 42 between the securement member 14 and the receiving channel 16 may be varied by altering the respective dimensions depending upon the desired degree of compression along the suture and the diameter of the suture used. With the variability, the height may be dimensioned to be no greater than 75% of the uncompressed diameter of the first portion 32 of suture in one variation or no greater than 90% of the uncompressed diameter of the first portion 32 in another variation. Additionally, the length of the suture compression interface 42 may also be varied through the anchor housing 12 to range from, e.g., 0.5 mm to 1.75 mm.

Different suture types may react differently to the compressive force imparted upon the first portion 32 of suture. For instance, braided sutures may splay out when compressed. Hence, given the native diameter of the suture used, the suture compression interface 42 height may be generally undersized but having the height improperly sized could potentially bulge or split the anchor housing 12 given the mechanical properties of the biodegradable or bioabsorbable materials used in forming the anchor assembly 10.

With the first portion 32 extending from the opening 16 about the periphery of the anchor housing 12 and along the securement member 16, the first portion 32 of suture 30 may then curve or bend through the suture receiving notch 22 within the anchor housing 12 such that a second portion of suture 34 passes through the first surface 36 of the anchor housing 12. The first portion 32 of suture may accordingly be orthogonal to or form an angle with the second portion 34 of suture, e.g., 60 degrees and up to 135 degrees. The suture 30 passes through the first surface 36 along a longitudinal axis of the anchor housing 12, forms a tortuous passageway around notch 22, is compressed along the suture compression interface 42, and then exits along the periphery of the anchor assembly 10.

To accommodate the second portion 34 of suture passing through the first surface 36, the anchor housing 12 may define a slot 40 along the first surface 36 which extends from the opening 16 towards a center portion of the anchor housing 12. The slot 40 may have a width ranging from, e.g., 40% to 50%, of a width or diameter of the anchor housing 12. When the securement member 14 is urged entirely into the receiving channel 26, the slot 40 may form an additional compression point with the notch 22 of the securement member 14 against the suture 30. The resulting compression and pinching of the suture 30 through the anchor housing 12 may result in a resistive pull force upon the suture of 0.125 lb and up to 3.5 lb. Moreover, even with the use of the biodegradable or bioabsorbable materials used in fabricating the anchor assembly 10, having the second surface 38 present a uniform and continuous surface helps to prevent or inhibit deformation of the anchor housing 12 upon application of the load to the suture 30 which also may help to enable a significant compression locking force at the interface. Additionally, the distal portion of the receiving channel 26 is also configured to limit expansion of the anchor housing 12 upon application of the load upon the suture 30.

As shown in the cross-sectional side view of FIG. 4A, the securement member 14 is illustrated prior to being inserted into the anchor housing 12. The length of suture 30 may be initially inserted into notch 22, as shown, and then securement member 14 may be advanced distally into the receiving channel 26 once the suture 30 is ready to be secured. As illustrated, the first portion 32 of suture may become compressed, as described, along the suture compression interface 42 formed between the upper surface of the securement member 14 and the apposed inner surface of the receiving channel 26 such that the first portion 32 of suture is compressed to a reduced thickness, e.g., up to 75% of the uncompressed diameter or up to 90% of the uncompressed diameter in another variation, which is less than the uncompressed suture diameter. The first portion 32 of suture may then pass through the notch 22 of securement member 14 such that the second portion 34 of suture passes through the first surface 36 of the anchor housing 12. The suture 30 may thus be compressed along the interface 42, secured via the tortuous passageway around notch 22, and also pinched between the notch 22 and slot 40 defined along the first surface 36.

FIG. 4C illustrates a cross-sectional side view of another variation where the suture 30 may be alternatively formed into a tortuous passageway between the distal end 50 of securement member 14 and the end of the receiving channel 26. The compressed first portion 32 of suture may thus extend along the entire length of the securement member 14 and then curve around the distal end 50 of the member 14 to then extend proximally back to the slot 40 where the second portion 34 of suture may then curve again to pass through the first surface 36 which may form yet another point for suture securement.

In deploying one or more of the tissue anchors described herein, a deployment instrument 60, as illustrated in the perspective view of FIG. 5, may be used. The deployment instrument 60 may generally comprise a handle 62 having an actuation plunger 64 attached. An elongate shaft 66 may extend from the handle 62 with a cartridge engagement housing 68 positioned at its distal end. An anchor deployment cartridge 70 may be removably attached to the engagement housing 68 for aligning the tissue anchors to be deployed. Each of the deployment cartridges 70 may contain a single tissue anchor or two or more tissue anchors aligned and engaged for deployment into the tissue region. Once the tissue anchors have been deployed or spent from the deployment cartridge 70, the cartridge 70 may be ejected from the engagement housing 68 and a new cartridge having additional tissue anchors may be engaged to the housing 68.

FIG. 6 illustrates a perspective view of one variation of a cartridge 70 which may house the one or more tissue anchors. The cartridge 70 may generally comprise a housing which contains an actuation mechanism for cinching the components of the anchor assembly 10 upon the suture 30. The cartridge 70 may thus define a tissue anchor retaining stage 76 into which the one or more tissue anchors may be aligned upon one another for deployment into the tissue region. The retaining stage 76 may form an opening into which the tissue anchors 10 may be placed and the stage 76 may further define a slot or opening 78 along its side to enable the passage or side-loading of the suture 30 into the tissue anchors within the cartridge 70. The cartridge 70 may further have a housing attachment 72 extending from the cartridge 70 and which is configured for secure reception by the engagement housing 68. The housing attachment 72 may further define an opening 74 into which an actuation mechanism may be advanced from the deployment instrument 60 for actuating a locking mechanism within the cartridge 70 for closing the tissue anchors upon the suture 30.

As the cartridge 70 may generally hold one or two tissue anchors, multiple cartridges 82 may be utilized with the deployment instrument 60, e.g., when suturing a portion of tissue with multiple interrupted sutures. One variation for facilitating the deployment of multiple tissue anchors may be seen in the perspective view of FIG. 7 which illustrates a cartridge assembly 80 configured as a carousel. Each individual cartridge 82 may be positioned within a cartridge slot 84 adjacent to one another in a circular manner. The cartridge assembly 80 may be rotated during use to facilitate the insertion of a cartridge into the engagement housing 68 of the deployment instrument 60.

FIGS. 8A to 8D illustrate an example of how the deployment instrument 60 may be used with the cartridge assembly 80. With the deployment instrument 60 ready for use to deploy tissue anchors into a tissue region, the engagement housing 68 may be advanced over a first cartridge 82 positioned upon the assembly 80, as shown in FIG. 8A, and placed upon a first cartridge 82, as shown in FIG. 8B. The engagement housing 68 may be locked to the first cartridge 82, as shown in FIG. 8C, whereupon engaged cartridge 82 and instrument 60 may be removed from the assembly 80 for anchor deployment, as shown in FIG. 8D. Once the tissue anchors within the first cartridge 82 have been used or spent, the first cartridge 82 may be ejected from the engagement housing 68 and a second cartridge may then be engaged from the assembly 80 for use.

The individual cartridges positioned upon the assembly may be specially marked or have some visual indicator to optionally indicate the first cartridge to be used and/or a terminal cartridge to be used, if so desired, where the initial cartridge or terminal cartridge may contain a single tissue anchor or specialized tissue anchor. The individual tissue anchors within the initial and/or terminal cartridge may also optionally incorporate some visual indicator, such as coloring, to indicate the initial tissue anchor (e.g., colored green) used in a procedure and/or a terminal tissue anchor (e.g., colored red) used in the procedure.

FIGS. 9A to 9F illustrate perspective views showing one example of how the cartridge 70 may be used with the deployment instrument 60 to suture a region of tissue. As shown in FIG. 9A, a first tissue anchor 90 having a first anchor housing 92 and first securement member 94 and a second tissue anchor 96 having a second anchor housing 98 and a second securement member 100 both in their unlocked configuration may be aligned atop one another within retaining stage 76, as shown in FIG. 9A. The length of suture 30 may be loaded through slot or opening 78 defined along the side of cartridge 70 and into the first and/or second securement members 94, 100. In variations of the cartridge 70 having at least two tissue anchors, each of the tissue anchors may be aligned relative to one another such that a first surface of each of the anchors is aligned in apposition to one another. That is, the surface of each anchor which contacts the tissue surface and having the suture exiting along the longitudinal axis of the anchor may be aligned in apposition to face each other within the cartridge 70 for deployment against the approximated tissue. Such an alignment may allow for the deployment of the tissue anchors in the same orientation to ensure that the suture passing between the anchors is aligned along each of the longitudinal axes of the anchors. This may prevent any rotation or moment being imparted upon the anchors when tensioned by the tissue.

With the suture 30 optionally passed through a tissue region T to be approximated, the second tissue anchor 96 may be secured against the length of suture 30 by actuating the plunger 64 such that an actuation member 102 is urged through the engagement housing 68 and into contact against a second sliding member 104. The distal end of actuation member 102 may be angled or tapered and the proximal end of second sliding member 104 may also be angled in a corresponding manner such that when the angled end of member 104 is pressed by actuation member 102, member 104 may slide distally into contact against the first anchor housing 98 to drive the anchor housing 98 and securement member 100 into securement upon the suture 30, as shown in FIG. 9B.

With the second anchor 96 secured upon the suture 30, anchor 96 may be removed from the retaining stage 76 of cartridge 70, as shown in FIG. 9C, for deployment against the tissue. With first tissue anchor 90 remaining within the retaining stage 76, the deployment instrument 60 may be optionally removed to another tissue location and the remaining suture length trimmed, if desired. Alternatively, once the second tissue anchor 96 has been removed from the cartridge 70, a length 108 of suture may be drawn through the cartridge 70 to a desired length, as shown in FIG. 9D, and the actuation member 102 may be further actuated to drive its distal end into contact with the first sliding member 106 which may also define an angled proximal end for engagement with the actuation member 102, as shown in FIG. 9E, to then lock the first tissue anchor 90 against the suture 30 for deployment against the tissue T. The first tissue anchor 90 secured to the suture 30 may then be removed from the retaining stage 76 of cartridge 70, as shown in FIG. 9F. The spent cartridge 70 may then be ejected from the deployment instrument 60 and another cartridge may be engaged to the engagement housing 68, if so desired.

In another variation, the cartridge 70 may optionally incorporate a cutting element (not shown for clarity) configured manually or automatically to cut the length of suture 108 extending between each of the anchors 90, 96. Such a cutting element may incorporate any number of severing mechanisms, e.g., blade, wire, energized element, etc., which may be actuated by the actuation member 102 or a separate actuation mechanism.

Another variation of the tissue anchor is illustrated in the perspective and top views of FIGS. 10A to 10F. While this variation shows a rotatable tissue anchor assembly 110, the rotational mating of the anchor housing 112 and securement member 114 utilizes the same suture compression interface and securement principles described herein for the other anchor variations. The securement member 114 may be formed into a circular and planar structure which may be received into the anchor housing into a correspondingly sized channel. As shown, the anchor housing 112 may be rotationally attached via a rotational pivot 116 to a portion of the securement member 114. The anchor housing 112 may define a peripheral opening 118 adjacent to the pivot 116 as well a slot, e.g., an arcuate slot 120, defined along the first surface 124. Similarly, securement member 114 may also define a suture receiving slot 122 configured in apposition to the arcuate slot 120. The second surface 126 of the anchor housing 112 may define an uninterrupted structure to improve the structural integrity of the anchor housing 112 when the suture is secured, as previously described.

The length of suture 30 may be initially loaded through the suture receiving slot 122 of securement member 114, as shown in FIGS. 10A to 10D. With suture 30 positioned within the slot 122, the suture 30 may be optionally aligned with or in proximity to the central axis of the securement member 114. The securement member 114 may then be rotated about pivot 116 such that the suture receiving slot 122 in securement member 114 and arcuate slot 120 along the first surface 124 of anchor housing 112 are brought into alignment with one another, as shown in FIGS. 10E and 10F.

As the securement member 114 is further rotated into peripheral opening 118, the first portion 32 of suture may become compressed along the suture compression interface 130 formed between the upper surface of securement member 114 and the inner surface of the opening 118, as shown in the perspective and top views of FIGS. 11A and 11B. The suture compression interface 130 may retain the same compressive height and interface compression characteristics as described herein.

Moreover, with the securement member 114 fully rotated into engagement within peripheral opening 118, the first portion 32 of suture compressed along the suture compression interface 130 may be further compressed or pinched along the second portion 34 of suture between the interface of slot 122 and slot 120 to provide for further securement to the suture 30, as shown in the perspective views of FIGS. 11C and 11D. Additionally, and as described herein, the suture passing through the first surface 124 may extend from the anchor housing 112 optionally aligned with the longitudinal axis of the anchor assembly 110. Because the first surface 124 is configured for presentation against the tissue surface, having the suture 30 extend along the longitudinal axis of the anchor may prevent or inhibit any rotation force or moment from being imparted upon the tissue anchor when the suture is tensioned by the tissue.

FIGS. 12A to 12C show side and cross-sectional side views of the tissue anchor to further illustrate the compression and tortuous path imparted upon the suture 30 when locked into securement. As shown, the suture 30 may be initially side-loaded into the suture receiving slot 122 and once securement member 114 has been fully rotated into engagement with anchor housing 112 through the peripheral opening 118, as shown in FIG. 12B, the suture 30 may be compressed accordingly along the first portion 32 of suture within the suture compression interface 130. The suture length may be further formed into a tortuous path through the slot 120 such that the second portion 34 of suture may also be further compressed or pinched between the interface of slot 122 and arcuate slot 120, as shown in the cross-sectional side view of FIG. 12C.

These tissue anchors may also have the same or similar dimensions and anchoring characteristics as described herein for other tissue anchor variations. Additionally, the tissue anchors may be fabricated from any of the materials as also described herein.

An example of how multiple tissue anchors may be deployed as illustrated in the perspective view of FIG. 13 where multiple anchors 140 (for instance, any of the anchor variations described herein) each having a length of suture passed through the tissue T and between a first anchor and a second anchor may be seen within the oral cavity OC of a patient. The tissue anchors may thus be used for a number of various procedures, e.g., closing the oropharyngeal tissue in a uvulopalatopharyngoplasty (UPPP) procedure, etc., not only in the oral cavity OC but elsewhere in the body where low-profile tissue anchors which are biodegradable or bioabsorbable may be used.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue anchor, comprising:
a securement member defining a suture receiving channel which opens along a side portion of the securement member; and
an anchor housing defining a receiving channel sized to receive the securement member within the anchor housing such that the suture receiving channel along the securement member is enclosed within the receiving channel when the securement member is secured within the anchor housing.

2. The anchor of claim 1 wherein the securement member which is adjustably slidable relative to the receiving channel such that the suture receiving channel is aligned with an opening defined along a first surface of the anchor housing for presentation against a tissue surface.

3. The anchor of claim 2 wherein the securement member and a compression surface along the receiving channel are spaced apart from one another and form a suture compression interface.

4. The anchor of claim 3 further comprising a suture having a first portion positioned along the suture compression interface and a second portion passed through the suture receiving channel and opening along the first surface, wherein the suture compression interface has a height which is sized to be relatively smaller than a diameter of the first portion of suture.

5. The anchor of claim 4 wherein the height of the suture compression interface is no greater than 75% of an uncompressed diameter of the first portion of suture.

6. The anchor of claim 4 wherein the suture compression interface is sized to create an interference fit between the first portion of suture and the anchor.

7. The anchor of claim 1 wherein the securement anchoring member and anchor housing are comprised of a dissimilar material.

8. The anchor of claim 1 wherein the tissue anchor is comprised of a biodegradable material.

9. The anchor of claim 1 wherein the anchor housing is configured into a circular shape.

10. The anchor of claim 1 wherein the anchor housing comprises a diameter of 3.5 mm to 5.0 mm.

11. The anchor of claim 1 wherein the anchor housing comprises a height of 1.5 mm to 3.0 mm.

12. The anchor of claim 3 wherein the suture compression interface has a length through the anchor housing ranging from 0.5 mm to 1.75 mm.

13. The anchor of claim 3 wherein the suture compression interface lies within a plane defined by the anchor housing.

14. The anchor of claim 1 wherein the securement member comprises an elongate structure linearly insertable into the receiving channel.

15. The anchor of claim 1 wherein the securement member comprises a structure rotatably insertable into the receiving channel

16. The anchor of claim 1 wherein the securement member is configured to retain a suture within the suture receiving channel.

17. The anchor of claim 16 wherein the suture is slidably retained within the suture receiving channel.

18. The anchor of claim 16 wherein movement of the suture relative to the suture receiving channel is inhibited when the securement member is secured within the anchor housing.

19. The anchor of claim 1 wherein the side portion is located proximal to a distal end of the securement member.

20. A tissue anchor, comprising:
a securement member defining a suture receiving channel which opens along a side portion of the securement member;
an anchor housing defining a receiving channel sized to receive the securement member within the anchor housing such that the suture receiving channel along the securement member is enclosed within the receiving channel when the securement member is secured within the anchor housing, and
wherein the securement member which is adjustably slidable relative to the receiving channel such that the suture receiving channel is aligned with an opening defined along a first surface of the anchor housing for presentation against a tissue surface.

21. The anchor of claim 20 wherein the securement member and a compression surface along the receiving channel are spaced apart from one another and form a suture compression interface.

22. The anchor of claim 21 further comprising a suture having a first portion positioned along the suture compression interface and a second portion passed through the suture receiving channel and opening along the first surface, wherein the suture compression interface has a height which is sized to be relatively smaller than a diameter of the first portion of suture.

23. The anchor of claim 22 wherein the height of the suture compression interface is no greater than 75% of an uncompressed diameter of the first portion of suture.

24. The anchor of claim 22 wherein the suture compression interface is sized to create an interference fit between the first portion of suture and the anchor.

25. The anchor of claim 20 wherein the securement anchoring member and anchor housing are comprised of a dissimilar material.

26. The anchor of claim 20 wherein the tissue anchor is comprised of a biodegradable material.

27. The anchor of claim 20 wherein the anchor housing is configured into a circular shape.

28. The anchor of claim 20 wherein the anchor housing comprises a diameter of 3.5 mm to 5.0 mm.

29. The anchor of claim 20 wherein the anchor housing comprises a height of 1.5 mm to 3.0 mm.

30. The anchor of claim 21 wherein the suture compression interface has a length through the anchor housing ranging from 0.5 mm to 1.75 mm.

31. The anchor of claim 21 wherein the suture compression interface lies within a plane defined by the anchor housing.

32. The anchor of claim 20 wherein the securement member comprises an elongate structure linearly insertable into the receiving channel.

33. The anchor of claim 20 wherein the securement member comprises a structure rotatably insertable into the receiving channel.

34. The anchor of claim 20 wherein the securement member is configured to retain a suture within the suture receiving channel.

35. The anchor of claim 34 wherein the suture is slidably retained within the suture receiving channel.

36. The anchor of claim 34 wherein movement of the suture relative to the suture receiving channel is inhibited when the securement member is secured within the anchor housing.

37. The anchor of claim 20 wherein the side portion is located proximal to a distal end of the securement member.

38. A method of locking a suture into an anchor, comprising:
positioning a suture within a suture receiving channel which opens along a side portion of a securement member;
adjusting a relative position of the suture relative to the securement member while retaining the suture within the suture receiving channel such that the suture is slidable within the suture receiving channel; and
advancing the securement member into a receiving channel defined within an anchor housing such that the suture receiving channel along the securement member is enclosed within the receiving channel and movement of the suture relative to the suture receiving channel is inhibited.

39. The method of claim 38 wherein the anchor housing defines a first surface for presentation against a tissue surface, a second surface opposite to the first surface, and a periphery between the first and second surfaces, the anchor housing defining the receiving channel within or along the periphery.

40. The method of claim 39 wherein advancing the securement member comprises sliding the securement member into the receiving channel such that the suture receiving channel is aligned with an opening defined along the first surface.

41. The method of claim 40 further comprising compressing the suture within or along a suture compression interface formed between the securement member and a compression surface along the receiving channel spaced apart from one another, wherein the suture compression interface has a height which is sized to be relatively smaller than a diameter of the suture.

42. The method of claim 41 wherein compressing the suture comprises compressing a first portion of the suture along the suture compression interface to a height no greater than 75% of an uncompressed diameter of the first portion of suture.

43. The method of claim 42 wherein compressing a first portion of suture comprises creating an interference fit between the first portion of suture and the anchor.

44. The method of claim 42 wherein compressing a first portion of suture comprises forming an orthogonal passageway of the suture through the anchor housing.

45. The method of claim 38 wherein the securement anchoring member and anchor housing are comprised of a dissimilar material.

46. The method of claim 38 wherein the anchor housing is comprised of a biodegradable material.

47. The method of claim 38 wherein advancing the distal end of the securement member comprises positioning a first portion of the suture and a second portion of the suture to define an angle relative to one another within the anchor housing.

48. The method of claim 47 wherein the angle between first portion and second portion ranges from 60 degrees to 135 degrees.

49. The method of claim 38 wherein the side portion is located proximal to a distal end of the securement member.
